(19) United States
(12) Patent Application Publication
(10) Pub. No.: US 2018/0146990 A1
(43) Pub. Date: May 31, 2018

(54) APPARATUS AND METHOD FOR ACCESSING THE SPINE AND PLACING PEDICLE SCREWS WITHOUT THE USE OF GUIDE-WIRES

(71) Applicant: MIS IP I Holdings LLC, Denver, CO (US)

(72) Inventors: James Manzanares, Kissimmee, FL (US); Gerald Schell, Bay City, MI (US); Jeffrey Schell, Denver, CO (US); Brandon Arthurs, Wilmington, NC (US); Scott Noble, Denver, CO (US); Ryan Arce, Denver, CO (US); Leighton LaPierre, Thornton, CO (US)

(73) Assignee: MIS IP I Holdings LLC, Denver, CO (US)

(21) Appl. No.: 15/826,244

(22) Filed: Nov. 29, 2017

Related U.S. Application Data
(60) Provisional application No. 62/427,374, filed on Nov. 29, 2016.

(51) Int. Cl. A61B 17/70

(52) U.S. Cl. CPC A61B 17/7082; A61B 5/04001

(57) ABSTRACT

The preferred embodiment of the present invention is an apparatus and operation for the placement of pedicle screws without the use of guide wires. The preferred embodiment of the present invention improves upon previous systems for the placement of pedicle screws by reducing the risks associated with the use of guide wires. The preferred embodiment of the present invention also enables the reduction of steps associated with the surgical procedure to place one or more pedicle screws.

This application claims the benefit of U.S. Provisional Patent Application 62/427,374 entitled “SYSTEM FOR ACCESSING THE SPINE AND PLACING PEDICLE SCREWS WITHOUT THE USE OF GUIDE-WIRES” filed on Nov. 29, 2016, the entire contents of which are incorporated herein by reference in its entirety for all purposes.

### FIELD OF THE INVENTION

The present invention relates to spinal surgery, and in particular, the surgical placement of pedicle screws without requiring the installation of a guide-wire prior to guide the placement of the screw.

### BACKGROUND OF THE INVENTION

Multi-segmental spinal fixation is an accepted surgical procedure in the treatment of disorders of the spine. Spinal fixation regularly involves the use of a series of pedicle screws and connecting rods to support the spine posteriorly. In order to create a spinal fixation, a structure, such as a rod, is fastened to two or more adjacent vertebral bodies through the use of pedicle screws. Pedicle screws, specially designed threaded fasteners, are carefully placed through both pedicles of the spine and into the vertebral body. This process is repeated for at least two adjacent vertebral bodies. A rod, spanning between pedicle screws on either side of the spinous process is affixed to the pedicle screws. Pedicle screws and rods are used in a spinal fusion procedure to provide stability and add extra support and strength to a spinal fusion area. This type of fixation is intended to prevent movement and allows the bone graft to heal as intended by the surgeon.

In a typical procedure involving posterior fixation of the spine, the steps associated with the placement of pedicle screws must be replicated at least four times—two per vertebral body in order to connect at least two adjacent vertebral bodies. These steps include those relevant to targeting the relevant portion of the vertebral body, accessing the relevant portion of the vertebral body, and preparing cortical bone holes for pedicle screw placement.

A series of steps associated with a typical procedure involving the placement of pedicle screws is the step of inserting an access needle. The access needle, commonly referred to as a “cannulated needle” or “Jamshidi needle,” is a needle used to initially establish a path, typically with imagery confirmation, to the target-site. The access needle has a cannula extending through the length of the needle, allowing a surgeon to advance a guide-wire through the cannula to the established pathway. The guide-wire, often referred to as a “Kirschner wire” or “K-wire,” allows the surgeon to advance dilators and other through the newly created pathway. These steps involve the placement of an access needle into the skin and traversing through the soft tissue to define the pathway to the relevant portion of a vertebral body as targeted by a surgeon. Once the access needle is placed, a guide-wire is placed through the cannula of the access needle which extends from the handle of the access needle to the tip of the access needle. The guide-wire is embedded into the vertebral body at the target-site. Upon embedding the wire into the vertebral body at the target-site, the needle can be removed, allowing the surgeon to advance increasing diameter dilators until a pathway is large enough to allow the surgeon to prepare the target-site and advance a pedicle screw along over the guide-wire to the target-site. The steps can take a surgeon only a few minutes, but more commonly it may take a surgeon up to 6 minutes or more. These processes must be repeated at least 4 times depending on the number of vertebral bodies the surgeon chooses to connect with a construct involving pedicle screws and rods. Thus, the placement of guide-wires for posterior fixation may take upwards of 24 minutes when performing a one-level fixation, which involves the fixation of two adjacent vertebral bodies. Each additional level of fixation adds upwards of 12 minutes.

Another step associated with the placement of pedicle screws in previously known techniques is dilation. Dilation generally involves the placement of one or more tubes, or dilators, in expanding succession over a guide-wire. Generally, a first dilator having an internal diameter similar to the diameter of a guide-wire is placed over the guide-wire, and then one or more subsequent dilators of increasingly larger internal diameters are placed over the first dilator. The dilators thereby incrementally expand the tissue surrounding the guide-wire to define a pathway for a pedicle screw to be placed into the vertebral body. The time taken to place the dilators typically requires 1-3 minutes per screw. During a typical procedure, which requires the placement of at least 4 screws, this step is repeated at least 4 times. Each additional level of fixation requires of upwards of six minutes. The time requirements associated with the placement of dilators burdens both surgeons and surgery facilities.

Another step associated with the placement of a pedicle screw in a typical procedure involves the separate process of placing an awl or an awl-probe or a tap. It will be appreciated that a tap is used to cut or form the female threading which a male threaded element may engage with. During this step, a surgeon places an awl into the cortical bone, creating a pilot hole in the cortical bone in preparation for placing a tap or a pedicle screw into the pilot-hole and through the pedicle. This extra step typically takes an additional 30-60 seconds of time.

In addition to the above discussed steps, a surgeon may optionally drill or tap a hole to further prepare the pathway for the advancement of a pedicle screw. A drill or tap is advanced through the outermost dilator or a cannulated drill or cannulated tap is advanced over the guide-wire. The drill or tap is then driven into the cortical bone to create a hole for a pedicle screw. This step typically requires approximately 1-3 minutes per screw placement.

### SUMMARY OF THE INVENTION

A known problem associated with pedicle screw and rod fixation is the high number of steps and resulting extended intraoperative times and increased radiation exposure associated with posterior spinal fusion. Each of the aforementioned steps require intraoperative time and additional radiation exposure and potentially increase the risk to a patient. Furthermore, the number of steps required for current spinal fusion procedures subjects surgeons to time demands and increased radiation exposure on the surgeons performing related procedures and the medical facilities hosting the procedures. As a result, patients requiring spinal fusion procedures are forced to wait longer periods of time to have a scheduled procedure.

It is an aspect of the present invention to limit the number of steps and/or the amount of intraoperative time and radiation exposure, thereby reducing potential risks associated with extended intraoperative times. By way of eliminating the need for an initial guide-wire, the present invention allows the placement of pedicle screws with reduced intraoperative time, and potentially reducing risk to the patient. At the heart of the present invention is the inventors' discovery that a number of risks and steps may be removed from surgical procedures associated with the placement of pedicle screws by eliminating the need for a guide-wire and sequential dilators. A risk associated with the use of guide-wire involves advancing a guide-wire too far into the vertebral body and through the anterior wall of the vertebral body. Major vascular structures, including the aorta and vena cava, lie generally anterior and proximal to the vertebral body. A wire that travels too far into and through the vertebral body risks puncturing these vascular structures. If a surgeon punctures the aorta with guide-wire, a known risk associated with previously known procedures, the surgeon must act with urgency to address the emergency situation of patient bleeding at a high rate. Puncturing the aorta results in the abdomen filling with blood flowing through the point of puncture of the vascular structure. This situation requires the spine surgeon's emergency collaboration with a general surgeon to create separate access pathway to the aorta, and further collaboration with a vascular surgeon to repair the puncture to the aorta. Alternately, if a spine surgeon punctures the vena cava with a guide-wire, the patient will likely die. The high rate of bleeding through a puncture of the vena cava would likely cause patient death due to blood loss before a general surgeon and vascular surgeon could collaborate with the spine surgeon to repair the vena cava.

It is an aspect of embodiments of the present invention to greatly reduce the potential for puncturing the aorta or vena cava by eliminating the need to advance a guide-wire into the operative space and into the vertebral body.

Moreover, the processes associated with insertion of an access needle and guide-wire are costly. To perform procedures involving guide-wires, a spine surgeon must use costly disposable access needles and guide-wires. A typical spinal fusion procedure generally requires the disposal of at least one guide-wire per screw, increasing cost per surgery. Spine surgeons occasionally attempt to re-use guide-wires. This unorthodox tactic increases risk of infection and potential complication rate for patients. Furthermore, the stresses placed upon a wire may reduce the structural integrity of the wire, and thereby reduce safety of its use in surgery.

It is an aspect of embodiments of the present invention to reduce the wasteful and cost intensive practice of the use and disposal of guide-wires associated with the use of pedicle screws.

An associated risk is that surgeons may over-tap a hole in preparation for the advancement of a pedicle screw. Over-tapping a hole results in a hole that may be too deep or too broad for the proper fixation of a pedicle screw. Over-tapping may lead to suboptimal fixation of a pedicle screw into a vertebral body.

Surgeons face risks associated with this step, because if a drill is not strictly and properly measured for the preparation of a hole for the advancement of the pedicle screw, the drill may traverse too deep into the vertebral body, risking improper fixation of the following pedicle screw, or worse, puncturing through the vertebral body and into the vascular structures anterior to the vertebral body.

If the steps of tapping or drilling are not performed optimally, the pedicle screw fixation will suffer, resulting from improper hole preparation. A suboptimal fixation may result in reduced fixation strength of the pedicle screw and increased risk of a pedicle screw pulling out of the vertebral body.

Another problem associated with the tapping and drilling steps for the insertion of a pedicle screw surrounds the risk of pedicle fracture. Tapping or drilling of the cortical bone of a vertebral body can transfer forces into the bone structure, resulting in fractures. Pedicle fractures may propogate into other parts of the vertebral body under compressive loads, as the spine bears the weight of the body. A spinal fracture such as a pedicle fracture can lead to deformities or bone spurs which may affect the nerve structures surrounding the spine. Thus, deformities or bone spurs may result in painful sensations felt by the patient, negatively affecting quality of life for the patient.

Certain embodiments of the present invention surround a system for the placement of a pedicle screw without a guide-wire, allowing a surgeon to advance a pedicle screw into a vertebral body in anticipation of final placement of a pedicle screw without the use of a guide-wire, thereby eliminating a number of risks and time-consuming steps associated with the use of guide-wire.

It will be appreciated by those skilled in the art, that a rigid body provides increased directional control and delivery through soft tissue because the guide wire, due to its small diameter, is more prone to bending than a pedicle screw. Thus, the present invention allows increased control of a pedicle screw, in contrast with a guide-wire, for establishing and delivering a pedicle screw to a target site.

Certain embodiments comprise an apparatus for the placement of a pedicle screw and related devices. Embodiments of the present invention allow for the combination of steps related to vertebral body targeting, soft tissue dilation, bone hole preparation and screw insertion in a streamlined manner into fewer steps as compared to current practice. The present invention thereby also minimizes the risks deriving from the higher number of separate, discrete steps in previously known procedures. For purposes of this disclosure, in reference to any apparatuses associated with the system, the term “proximal” shall mean closer to a surgeon's torso and the term “distal” shall mean farther away from the surgeon's torso unless otherwise explicitly stated.

Certain embodiments of the present invention surround a method for the placement of a pedicle screw without requiring the use of a guide-wire or successive dilators to accurately deliver a pedicle screw to the target-site of a pedicle. A surgeon, using the apparatus of the present invention loads a pin through the first end of the apparatus, and advances the pin through apparatus until the pin extends from the tip of a cannulated pedicle screw which extends from the second end of the apparatus. The surgeon, having made an incision, advances the second end of the apparatus toward the target-site using fluoroscopic or other navigation methods until the pin contacts the target-site. Then embedding the pin into the target-site by tapping or striking the first end of the apparatus. Then advancing the pin further, preferably through the pedicle and into an attached vertebral body. Once the pin is advanced through the pedicle and into the vertebral body, stimulating the pin with an electrical signal for neuromonitoring. After confirming that the pathway is as desired, retracting the pin from the pathway, and advancing the pedicle screw along the pathway by rotating the driver handle. Alternatively, the surgeon may prefer to advance the pedicle screw prior to the retraction of the pin from the pathway.

In certain embodiments, a method for the placement of a pedicle screw may further comprise the locking and unlocking the pin position in relation to the tip the cannulated pedicle screw.

These and other advantages will be apparent from the disclosure of the inventions contained herein. The above-described embodiments, objectives, and configurations are neither complete nor exhaustive. As will be appreciated, other embodiments of the invention are possible using, alone or in combination, one or more of the features set forth above or described in detail below. Further, this Summary is neither intended nor should it be construed as being representative of the full extent and scope of the present invention. The present invention is set forth in various levels of detail in this Summary, as well as in the attached drawings and the detailed description below, and no limitation as to the scope of the present invention is intended to either the inclusion or non-inclusion of elements, components, etc. in this Summary. Additional aspects of the present invention will become more readily apparent from the detailed description, particularly when taken together with the drawings, and the claims provided herein.

### BRIEF DESCRIPTION OF THE DRAWINGS

FIG. 1A—A front view of an apparatus of certain embodiments

FIG. 1B—A perspective view of an apparatus of certain embodiments

FIG. 2A—A perspective view of a handle of certain embodiments

FIG. 2B—A front cross-sectional view of a handle of certain embodiments

FIG. 2C—A perspective view of a handle of certain embodiments

FIG. 3A—A front view of a dilator of certain embodiments in a closed configuration

FIG. 3B—A front cross sectional view of a dilator of certain embodiments

FIG. 3C—A front view of a dilator of certain embodiments in an open configuration

FIG. 4—A perspective view of a retainer sleeve of certain embodiments

FIG. 5A—A perspective view of a driver of certain embodiments

FIG. 5B—A close-up perspective view of an end of a driver

FIG. 6A—A front view of a pedicle screw of certain embodiments

FIG. 6B—A close up view of a tip of a pedicle screw of certain embodiments

FIG. 6C—A perspective view of a pedicle screw of certain embodiments

FIG. 6D—A front view of a pedicle screw of certain embodiments

FIG. 7A—A perspective view of a pin of certain embodiments

FIG. 7B—A side view of a pin of certain embodiments

FIG. 7C—A side view of a pin of certain embodiments

FIG. 7D—A cross-sectional view of a pin of certain embodiments in a locked configuration

FIG. 7E—A cross-sectional view of a pin of certain embodiments in an unlocked configuration

FIG. 8—An exploded view of an apparatus of certain embodiments

FIG. 9—A diagrammatic view of a method of certain embodiments

### DETAILED DESCRIPTION OF VARIOUS EMBODIMENTS

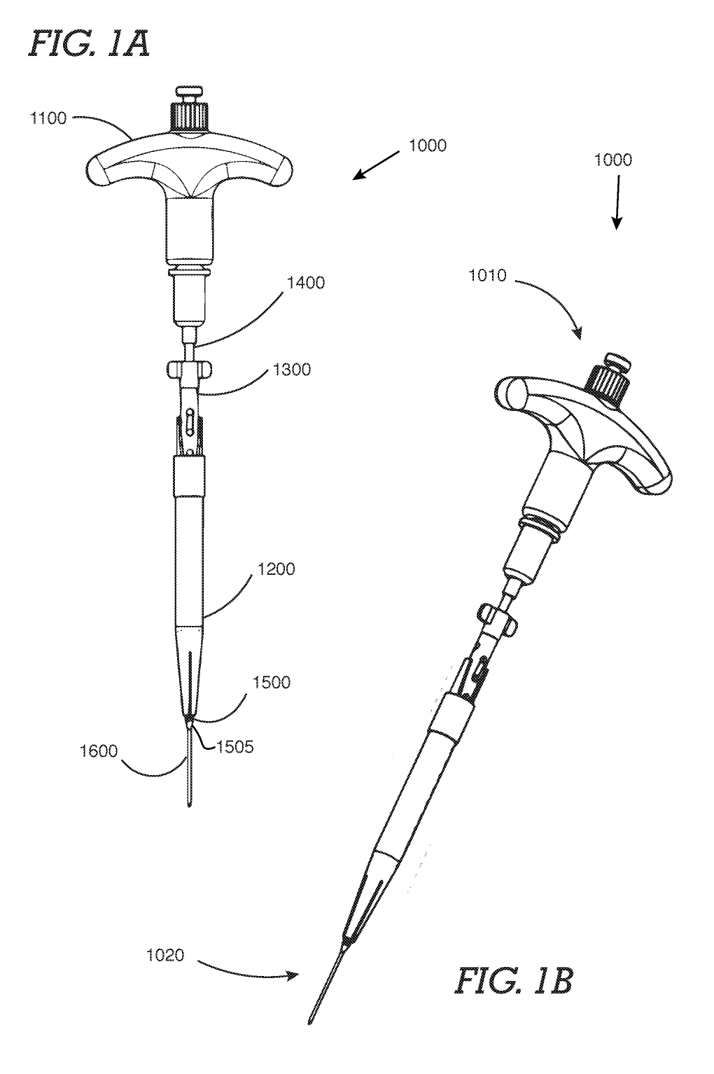

In certain embodiments as shown in FIG. 1A and FIG. 1B, an apparatus 1000 comprises a handle 1100 affixed to a first end 1010 of the apparatus, a dilator 1200, a retainer sleeve 1300, and a driver 1400. The apparatus is configured to deliver a pedicle screw 1500, through a second end 1020 of the apparatus to a target site. In certain embodiments, the tip 1505 of a pedicle screw extends outward from the second end 1020 of the apparatus, at a distal portion of the dilator 1200.

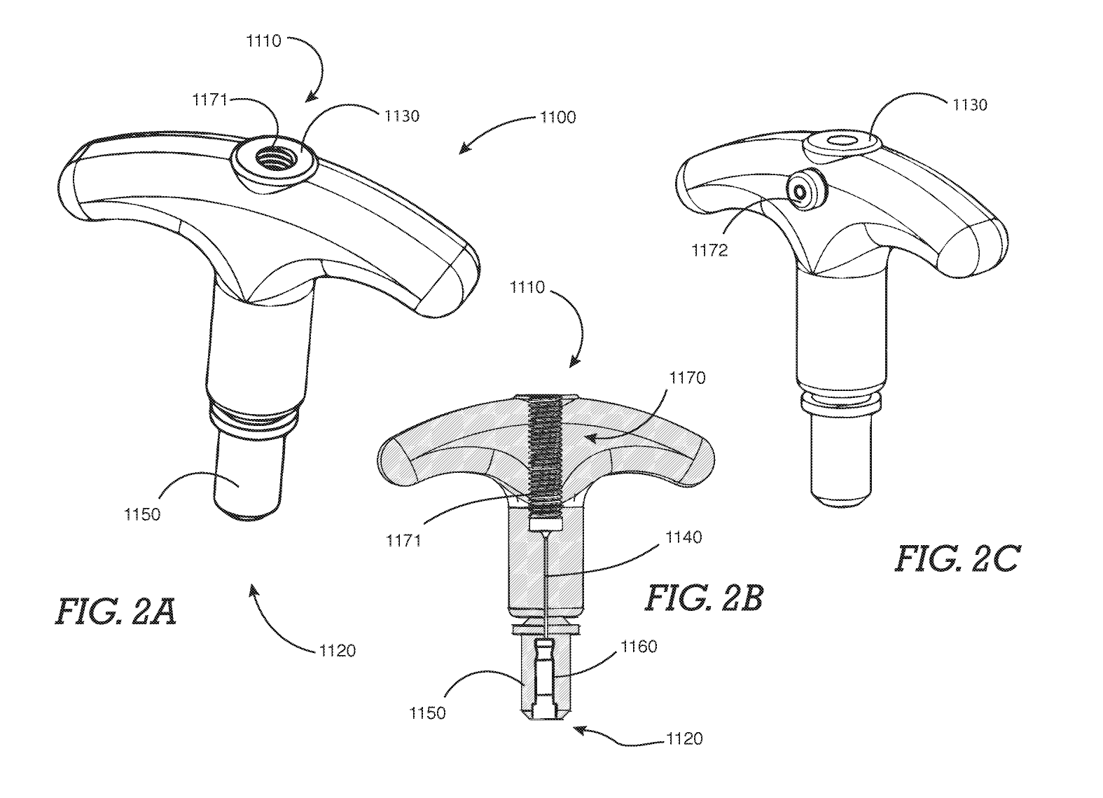

Certain embodiments, as shown in FIG. 2A and FIG. 2B, comprises an impact-plate 1130 at a first end 1110 of the handle to allow striking the handle with a hammer or other impact tool to advance the apparatus and thereby a pedicle screw. In certain embodiments, a second end 1120 of the handle comprises a driver retention feature 1150. The driver retention feature 1150 is configured to mate with and retain the first end of a driver (not shown) when assembled together. In certain embodiments, a handle 1100 further comprises a pathway extending through the first end 1110 and the second end 1120 of the handle. In certain embodiments, a driver retention feature 1150 further comprises a keyed feature 1160, while in some embodiments the driver retention feature 1150 further comprises a quick-release mechanism for the quick connection and disconnection of a handle 1100 from a driver 1400. In certain embodiments, the handle comprises a depth controller 1170 which acts to retain or controllably adjust the depth of a pin 1600 (FIG. 1A). In certain embodiments, a depth controller 1170 comprises a threaded feature 1171 as shown in FIG. 2A, while other embodiments comprise a ratchet lock 1172 as shown in FIG. 2C.

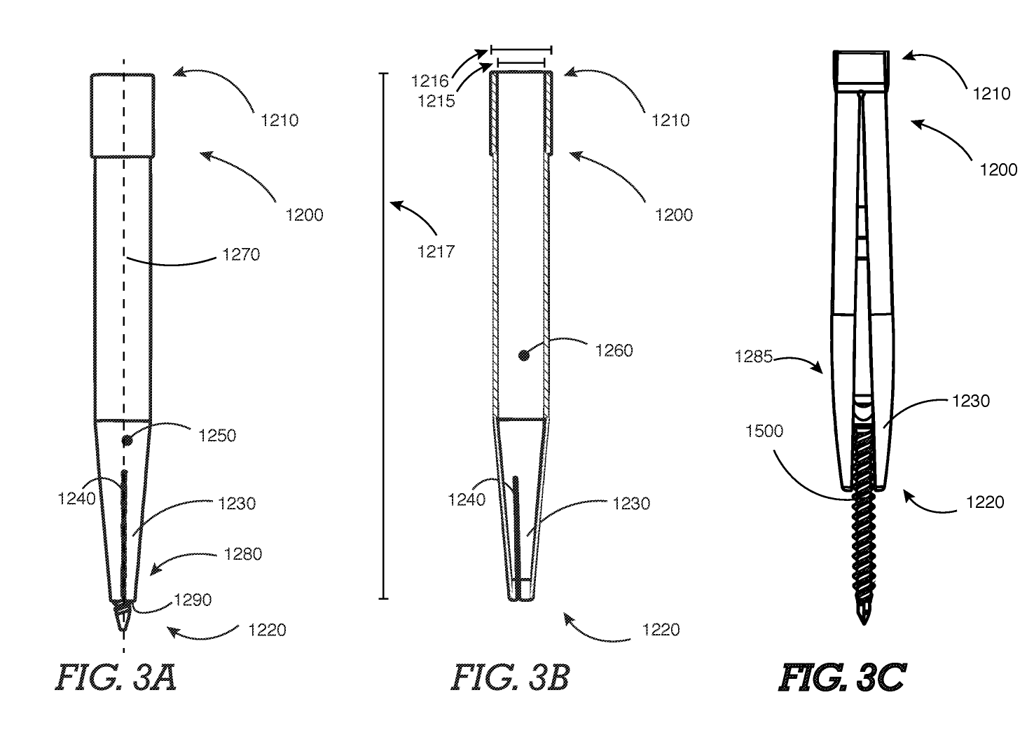

Certain embodiments, as shown in FIG. 3A, FIG. 3B, and FIG. 3C, comprise a dilator 1200 having an open first end 1210 having a internal diameter 1215, and a second end 1220 comprising a plurality of petals 1230. The internal diameter 1215 of the dilator is typically configured to accept a pedicle screw 1500 placed axially through the first end 1210 of the dilator. The petals 1230 are separated by slits 1240 which extend from the outer surface 1250 to the inner surface 1260 of the dilator. The slits 1240 extend from the second end 1220 of the dilator toward the first end 1210 of the dilator. It may be desired for the slits 1240 to run parallel to a central axis 1250 of the dilator. Prior to pedicle screw delivery, the dilator 1200 and petals 1230 are in a closed configuration 1280, allowing a surgeon to establish a pathway to the target-site. Once apparatus is at the target-site, and the pedicle screw is advanced, the second end 1220 of the dilator expands to allow the pedicle screw 1500 to pass through the second end 1220 of the dilator. As the pedicle screw passes through the second end 1220 of the dilator, the petals 1230 expand radially outward into an open configuration 1285. In certain embodiments, a dilator 1200 has an internal diameter 1215 of 15.5 mm, an outer diameter 1216 of 17.5 mm and a length 1217 of 150 mm.

Certain embodiments, shown in FIG. 3A-FIG. 3C, comprise a dilator 1200 further comprising an aperture 1290 at a second end 1220 of the dilator. The aperture 1290 of such embodiments is configured to allow the passage of portion of a pedicle screw 1500.

In certain embodiments, shown in FIG. 3A-FIG. 3C, a dilator 1200 comprises four slits 1240, each of which is 1 mm wide and 45 mm long, parallel with the central axis 1270 of the dilator 1200. In varying embodiments of the invention, a dilator 1200 may incorporate as few as two slits 1240 or as many as eight slits 1240. However, it will be further appreciated that embodiments may comprise a dilator 1200 having more than eight slits 1240 while keeping with the scope and spirit of the present invention. It will be appreciated that a slit 1240 of varying embodiments may comprise lengths longer and shorter than explicitly disclosed within the present application while remaining within the scope and spirit of the present invention. It will be further appreciated that a slit 1240 as disclosed is configured in relation to the material properties and dimensionalities of the dilator 1200 of the present invention to allow the passage of a pedicle screw 1500 without permanent deformation of the dilator 1200.

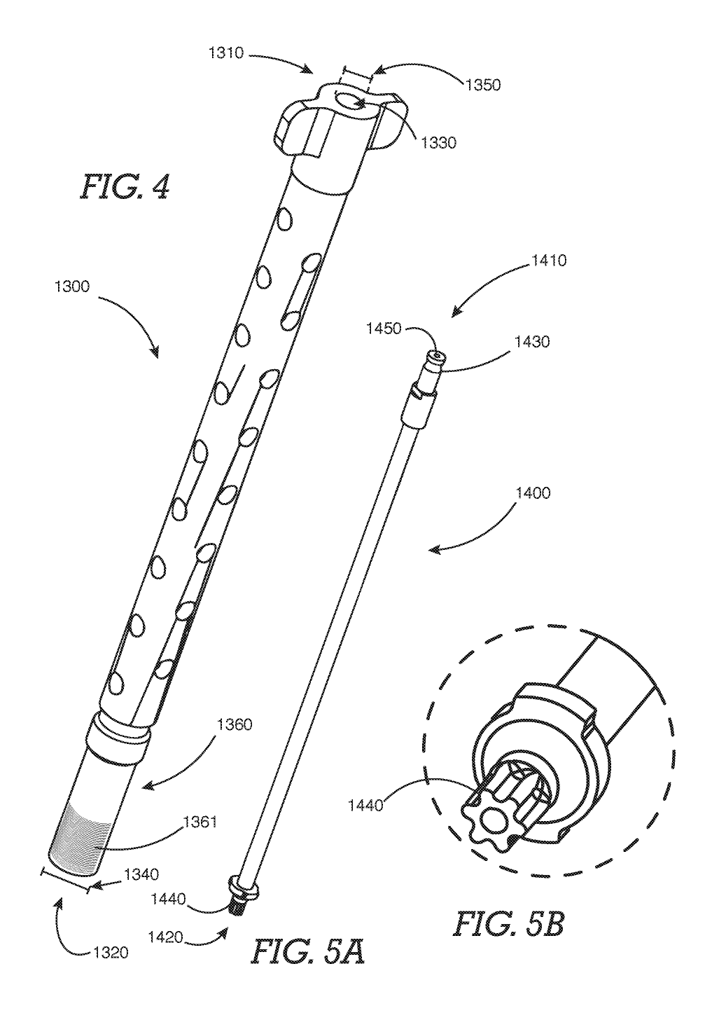

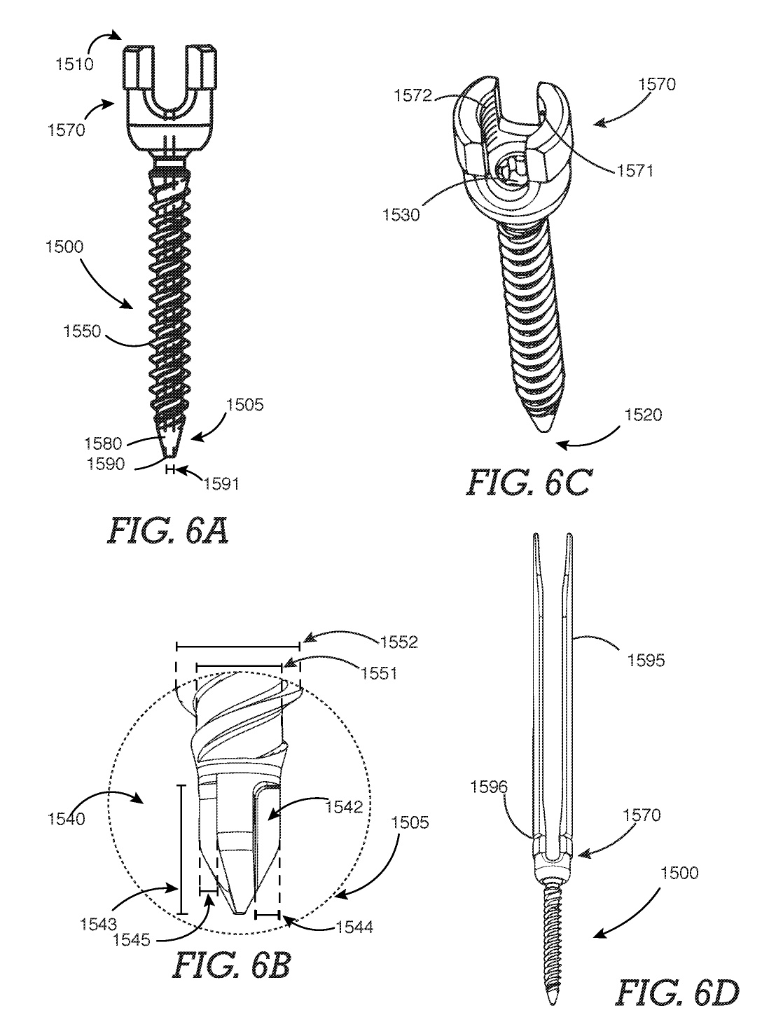

Certain embodiments, shown in FIG. 4, comprise a retention sleeve 1300 having a pathway 1330 passing from a first end 1310 to a second end 1320 of the retention sleeve 1300. The outer diameter 1340 of the retention sleeve is typically configured to be less than the internal diameter 1215 of a dilator (FIG. 3B). The pathway 1330 has an inner diameter 1350. In certain embodiments, a retention sleeve 1300 further comprises an attachment feature 1360 at the second end 1320 of the retention sleeve. An attachment feature 1360 of certain embodiments is configured to mate with and retain a pedicle screw. In certain embodiments, an attachment feature 1360 comprises threaded features, such as for the engagement with threaded features commonly found on an internal surface of a first end of a pedicle screw. In certain embodiments, the retainer sleeve 1300 retains the pedicle screw 1500 (FIG. 6A and FIG. 6B) by engaging with the threads 1575 located on the interior surface of the head of the pedicle screw, often referred to as the tulip 1570. In certain embodiments, the apparatus is configured to transfer impact forces applied axially through the apparatus to the tulip 1570 of the pedicle screw instead of the keyed feature 1530 of the pedicle screw.

It will be appreciated that in certain embodiments, a retainer sleeve 1300 comprises the towers 1595 of a pedicle screw, seen in FIG. 6D. A tower 1595 of a pedicle screw is an extension of the tulip 1570. A tower 1595 of certain embodiments are threadably detachable from the tulip 1570, while others are welded or otherwise affixed to the tulip 1570 of the pedicle screw. In such embodiments, it will be appreciated by those skilled in the art, a tower 1595 may be detached from the tulip 1570 by breaking the tower 1595 at an intended separation point 1596 by applying a moment to the tower 1595.

Certain embodiments, as shown in FIG. 5A-FIG. 5B, comprise a driver 1400 having a first end 1410 with a first keyed feature 1430, and a second end 1420 with a second keyed feature 1440. In certain embodiments, the driver 1400 further comprises a pathway 1450 extending from the first end 1410 of the driver to the second end 1420 of the driver. The first keyed feature 1430, is configured to mate with the driver retention feature 1150 and the keyed feature 1160 of the handle (FIG. 2B) to retain the driver 1400 in an axial direction and to translate rotative motion of the handle 1100 to rotative motion of the driver 1400. The second end 1420 of the driver comprises second keyed feature 1440. Certain embodiments of a second keyed feature 1440 are configured to mate with a first end of a pedicle screw 1500 (FIG. 6A) to allow rotational fixation between the driver 1400 and the pedicle screw 1500. In certain embodiments, as shown in FIG. 5B, a second keyed feature 1440 comprises a hexalobe profile.

Certain embodiments, as shown in FIG. 6A-FIG. 6C, comprise a pedicle screw 1500 having a first keyed feature 1530 at a first end 1510, and a tip 1505 with a hole-starting feature 1540 at a second end 1520 of the pedicle screw. The hole-starting feature 1540 allows the advancing of the pedicle screw 1500 into a target site without the need for preparation of the target site by way of steps and tools such as guide-wire, awling, probing, pre-drilling or tapping. The hole-starting feature 1540 of certain embodiments, shown in FIG. 6A and FIG. 6B comprise a non-threaded elongated tip 1505 protruding from the second end 1520 of the pedicle screw 1500. The hole-starting feature 1540 typically has a diameter 1551 less than or equal to the minor diameter 1551 of the threads. It will be appreciated that the diameter 1551 of the hole-starting feature 1540 may vary from the distal end of the tip 1505 to a more proximal end of the tip 1505. In certain embodiments, the hole-starting feature 1540 further comprises a recess 1542 machined radially into the hole-starting feature 1540. In certain embodiments, a recess 1542 tapers radially outward to where the tip 1505 meets the threads 1550. In certain embodiments, the threads 1550 are of a self-tapping thread type, thereby negating the need for the tapping of a hole prior to advancing the pedicle screw 1500 into the target site. It will be appreciated that a recess 1542 may be used as what is commonly referred to as a “chip-breaker” by those skilled in the art. These recesses 1542 decrease the amount of strain the bone is subjected to when the pedicle screw 1500 is advanced. These recesses 1542 remove small amounts of material from the hole initially formed in the target site, thereby decreasing the amount of material the pedicle screw 1500 must displace when advanced. In certain embodiments, a recess 1542 comprises a length 1543 of 1-3 mm, a depth 1544 of up to 50% of the major diameter 1552 in depth, and a width 1545 of 30% of the major diameter 1552. In certain embodiments, a recess 1542 extends the length of the hole-starting feature 1540 and into the threads 1550.

In certain embodiments, a hole-starting feature 1540 works in concert with the threads 1550, such as self-tapping threads of a pedicle screw. It will be appreciated to those skilled in the art that self-tapping threads surround screw threads which are configured to cut threads into a substrate as the screw is advanced. Self-tapping threads 1550 are incorporated into the pedicle screw 1500 as helical machined features along a portion the shaft of the screw. In such embodiments, the threads 1550 act in concert with the hole-starting feature 1540 by generating their own mating thread path. In certain embodiments, the tip 1505 is initially advanced by way of axial impact, for instance by striking an impact plate 1130 (FIG. 2A). Advancing the hole-starting feature 1540 into the cortical bone prepares a pathway for the threads 1550 to engage with the bone structure of the vertebral body. In certain embodiments a pedicle screw 1500 comprises threads 1550 which taper toward the tip 1505 of the pedicle screw. In certain embodiments, a pedicle screw 1500 comprises threads 1550 having a minor diameter 1551 between 4-8 mm (in) and a major diameter 1552 of 5-9 mm (in). In certain embodiments a pedicle screw 1500 comprises Titanium, 6Al 4V ELI per ASTM F136. Certain embodiments of a pedicle screw 1500, shown in FIG. 6A, comprise an internal surface 1571 at a first end 1510 of the pedicle screw, which comprises threaded features 1572.

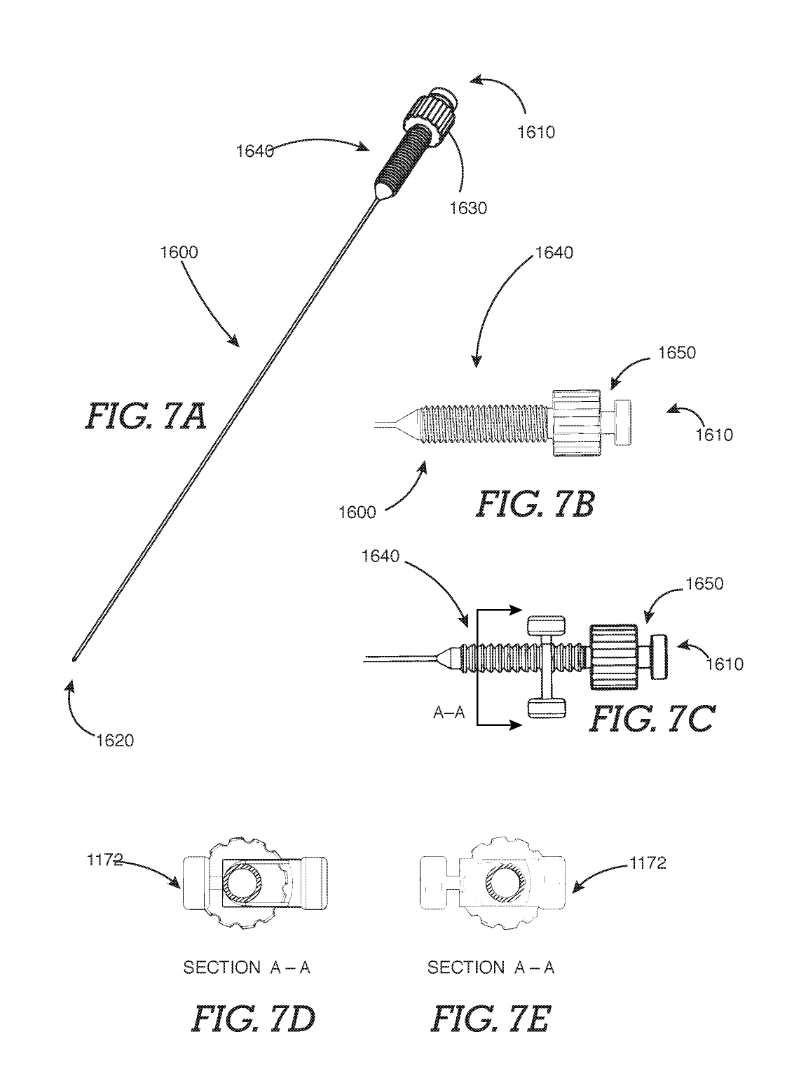

Certain embodiments of the present invention, shown in FIG. 7A-FIG. 7E, comprise a pin 1600 configured to be disposed through a pathway 1140 (FIG. 2B) of a handle. A pin 1600 of certain embodiments comprises a first end 1610 having a knob 1630 allowing the rotary indexing of the pin 1600 to incrementally advance the pin 1600. In certain embodiments, a first depth controller 1640 of the pin interfaces with a depth controller 1170 of the handle to provide incrementally controllable depth settings for the pin 1600. In certain embodiments, a depth controller 1640 of the pin and a depth controller 1170 (FIG. 2B) of the handle comprise mating threaded features, as shown in FIG. 7A. In other embodiments, a depth controller 1640 of the pin and a depth controller 1170 (FIG. 2B) of the handle comprise a ratchet and pawl system as shown in FIG. 7C, rack and pinion or other incrementally adjustable components appreciated by those skilled in the art. In certain embodiments, a handle 1100 having a ratchet and pawl system further comprises a locking feature 1170 (FIG. 2C) to allow a user to lock a pin 1600 in place, or unlock it to allow the advancing or retraction of the pin 1600 as desired. In certain embodiments, the pin 1600 further comprises a connection feature 1650 at a first end 1610 of the pin. It will be appreciated that a connection feature 1650 allows the connection of various instruments to provide axial tension on the pin 1600 to assist in retraction. In certain embodiments, it may be desired to configure the connection feature 1650 to mate with a slide-hammer, commonly referred to as a “slap-hammer” in the surgical field. In certain embodiments, shown in FIG. 2C, a handle 1100 further comprises a locking feature 1180 configured to retain a pin 1600 (FIG. 7C) at a desired depth of insertion. In certain embodiments, as shown in FIG. 7D and FIG. 7E, the locking feature 1180 further comprises a ratchet lock 1172. When in a locked configuration (FIG. 7D), the ratchet lock 1172 engage the locking feature 1180 and prevents the slidable movement of the pin 1600. When in an unlocked configuration (FIG. 7D) the ratchet lock 1172 allows the slidable movement of the pin 1600.

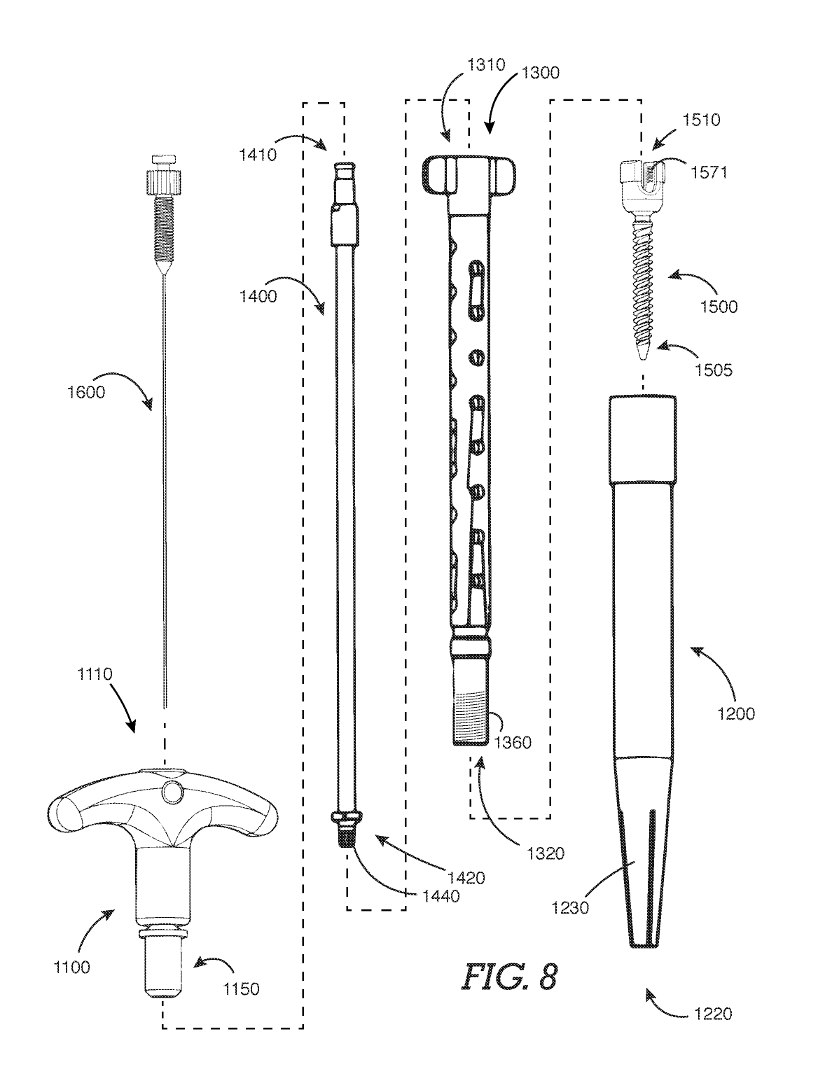

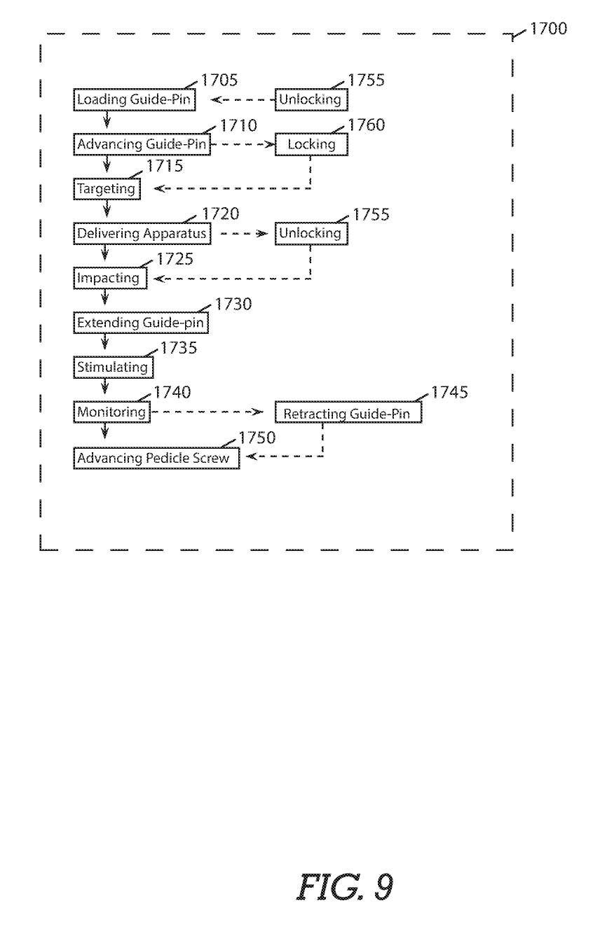

Certain embodiments of the present invention, shown in FIG. 8, comprise a retainer sleeve 1300 having a threaded attachment feature 1360 at a second end of the retainer sleeve engaged with a threaded feature 1572 on an internal surface 1571 at the first end 1510 of a pedicle screw. A driver 1400 disposed axially within a retainer sleeve 1300 such that the first end 1410 of the driver is proximate to the first end 1310 of the retainer sleeve, has a keyed feature 1440 at a second end 1420 of the driver engaged with a keyed feature 1530 (FIG. 6C) at a first end 1510 of the pedicle screw. The retainer sleeve 1300, is disposed within a dilator 1200 such that the second end 1320 of the retainer sleeve is proximate to the second end 1220 of the dilator. A handle 1100 is affixed to the driver 1400 such that the first end 1410 of the driver is retained within the driver retainer feature 1150 of the handle. The tip 1505 of a pedicle screw extends through an aperture 1290 at the second end 1220 of the dilator. The apparatus 1000 is directed toward the target site, and when tip 1505 of the pedicle screw is advanced into the target site, the pedicle screw 1500 advances out from the second end 1220 of the dilator. As the pedicle screw 1500 advances out from the second end 1220 of the dilator, the petals 1230 of the dilator expand radially outward. Thus, the dilator 1200 transitions from a closed configuration 1280 (FIG. 3A) to an open configuration 1285 (FIG. 3C) allowing the pedicle screw 1500 to fully advance into the target site. The pin, when disposed through the first end 1110 of a handle, traverses through the handle 1100, through the driver 1400, through the retainer sleeve 1300, through the dilator 1200 and through a pathway 1580 (FIG. 6A) of the pedicle screw where it extends out of an aperture 1590 (FIG. 6A) at the second end 1520 of the pedicle screw. Certain embodiments of a method 1700 for placing of a pedicle screw comprise the steps of: loading 1705 a pin into a first end of an apparatus for placing a pedicle screw; advancing 1710 the pin until the tip of the pin extends from the tip of the pedicle screw at the second end of the apparatus; targeting 1715 a target site; delivering 1720 the second end of the apparatus to the target-site; impacting 1725 the first end of the apparatus to embed the tip of the pin and/or hole-starting feature into the target site; extending 1730 the pin until the pin extends into the target site a desired distance; stimulating 1735 a first end of the pin with an electrical signal; monitoring 1740 using standard intraoperative neurophysiological monitoring procedures; retracting 1745 the pin; and advancing 1750 the pedicle screw into the target site.

Certain embodiments of a method 1700 for placing of a pedicle screw comprise the steps of: unlocking 1755 a handle, loading 1705 a pin into a first end of an apparatus for placing a pedicle screw; advancing 1710 the pin until the tip of the pin extends from the tip of the pedicle screw at the second end of the apparatus; locking 1760 the handle thereby locking the pin in place; targeting 1715 a target site; delivering 1720 the second end of the apparatus to the target-site; impacting 1725 the first end of the apparatus to embed the tip of the pin and/or hole-starting feature into the target site; unlocking the handle 1755 allowing the free movement of the pin; extending 1730 the pin by tapping the first end of the pin until the pin extends into the target site a desired distance; stimulating 1735 a first end of the pin with an electrical signal; monitoring 1740 using standard intraoperative neurophysiological monitoring procedures; retracting 1745 the pin; and advancing 1750 the pedicle screw into the target site. In certain embodiments, it may be desired to omit the step of retracting 1745 the pin, thereby advancing 1750 the pedicle screw with the pin in place.

While various embodiments of the present invention have been described in detail, it is apparent that modifications and alterations of those embodiments will occur to those skilled in the art. However, it is to be expressly understood that such modifications and alterations are within the scope and spirit of the present invention. Further, the inventions described herein are capable of other embodiments and of being practiced or of being carried out in various ways. In addition, it is to be understood that the phraseology and terminology used herein is for the purposes of description and should not be regarded as limiting. The use of “including,” “comprising,” or “adding” and variations thereof herein are meant to encompass the items listed thereafter and equivalents thereof, as well as, additional items.

What is claimed is:

1. An apparatus for the placement and advancement of a pedicle screw comprising:
a dilator having a first portion consistent with a first end having a substantially consistent cross-section with an internal diameter, a second portion consistent with a second end having a tapered profile, and a pathway therethrough;
a first petal and a second petal longitudinally extending along a portion of the length of the dilator and consistent with the second end of the dilator;
a first slit on a first side of the first petal and a second slit on a second side of the first petal, and the second slit separating the first petal from the second petal, and the slit in communication with the second end of the dilator;
a retainer sleeve having a first end, a second end and a pathway therebetween, the second end of the retainer sleeve having threaded features on the external surface, and the retainer sleeve having an internal diameter, and an external diameter which is less than the internal diameter of the dilator;
a driver having a first end and a second end, the first end of the driver having a first keyed feature and the second end having a second keyed feature, and an external diameter which is less than the internal diameter of the retainer sleeve;
a handle having an impact-plate at a first end of the handle, and a driver retention feature at a second end of the handle, the driver retention feature configured to receive the first keyed feature of the driver,
wherein the retainer sleeve is configured to axially mate with the dilator by sliding the second end of the retainer sleeve through the first end of the dilator,
the driver is configured to axially mate with the retainer sleeve by sliding the second end of the driver through the first end of the retainer sleeve and the second keyed feature of the driver is configured to mate with a keyed feature of a pedicle screw,
wherein the second keyed feature of the driver is configured to mate with a keyed recess in a first end of a pedicle screw held,
wherein the retainer sleeve threaded features are configured to mate with threaded features on an internal surface of a tulip of a pedicle screw thereby retaining the pedicle screw, and
wherein the advancement of the pedicle screw through second end of the dilator results in the radial expansion of the petals allowing the placement of the pedicle screw.

2. The apparatus of claim 1, wherein the second end of the dilator comprises an aperture aligned with the central axis of the dilator, wherein the aperture is configured to expose a tip of a pedicle screw disposed within the apparatus.

3. The apparatus of claim 1, wherein the handle further comprises a pathway extending from the first end of the handle to the second end of the handle.

4. The apparatus of claim 1, wherein the dilator further comprises and fourth petal; and the petals having slits between adjacent petals.

5. The apparatus of claim 1, further comprising a pedicle screw having a self-tapping feature.

6. The apparatus of claim 5, wherein the pedicle screw further comprises an axial pathway extending from a first end of the pedicle screw to the second end of the pedicle screw.

7. A method for the delivery of a pedicle screw to a target site comprising:
loading a pin into an apparatus;
advancing the pin until a tip of the pin extends from a distal end of the apparatus;
targeting the target-site;
delivering the apparatus to the target site such that a distal end of the apparatus contacts the target-site;
impacting a proximal end of the apparatus to embed the distal end of the apparatus into the target-site;
extending the pin a desired distance;
stimulating the pin with an electrical signal;
monitoring using standard intraoperative neurophysiological monitoring procedures;
and advancing a pedicle screw into the target-site.

8. The method of claim 7, wherein the step of loading the pin is preceded by a first step of unlocking the handle of the apparatus;
the step of advancing the pin is followed by a step of locking the apparatus; and
the step of delivering the apparatus is followed by a second step of unlocking the apparatus.

9. The method of claim 7, wherein the step of monitoring is followed by a step of retracting the pin, prior to the step of advancing the pedicle screw.
